(19) United States
(12) Patent Application Publication
(10) Pub. No.: US 2005/0208466 A1
(43) Pub. Date: Sep. 22, 2005

(54) Method of selective isolation or visualization of target cells differentiated from embryonic cells or kit for visualization

(75) Inventors: Kenichiro Kosai, Gifu-shi (JP); Tomoyuki Takahashi, Gifu-shi (JP)

Correspondence Address: APEX JURIS, PLLC, 13194 EDGEWATER LANE NORTHEAST, SEATTLE, WA, 98125, US

(73) Assignee: Nagoya Industrial Science Research Institute, Aichi (JP)

(21) Appl. No.: 10/518,861

(22) PCT Filed: Jun. 13, 2003
(86) PCT No.: PCT/JP03/07536

§ 371 Date: Mar. 21, 2005

(30) Foreign Application Priority Data
Jun. 17, 2002 (JP) 2002-175231

(51) Int. Cl. C12Q 1/00; C12Q 1/68; C12N 5/08; C12N 15/85

(52) U.S. Cl. 435/4; 435/6; 435/455; 435/366

(57) ABSTRACT

A method for selectively isolating or visualizing a target cell differentiated from an embryonic stem cell, which comprises transferring a first recombinant DNA in which a first promoter, a gene having recombinase recognition sequences on both ends, and a selective marker gene of a target cell differentiated from an embryonic stem cell are arranged in this order from a 5′ side, and the first promoter makes the selective marker gene express, and a second recombinant DNA in which a second promoter specifically expressing in a target cell differentiated from an embryonic stem cell, and a recombinase-expressing gene are arranged in this order from a 5′ side, respectively into an embryonic stem cell. A kit for isolation or visualization, which comprises the first vector for transferring a gene containing a first DNA and the second vector for transferring a gene containing a second DNA.

### TECHNICAL FIELD

The present invention relates to a method which can effectively and assuredly select and isolate, or visualize target cells differentiated embryonic stem cells and a kit for isolation or visualization used therefor.

### BACKGROUND FIELD

An embryonic stem (ES) cell (hereinafter, also referred to as ES cell) is a cell separated from a primary embryo, and this has such totipotence that the cell can be differentiated into any organ or cell such as hemocyte, cardiac muscle, skeletal muscle and nerve ones by manipulating the culturing system, development of study in the field of biology such as embryology, and advanced medicine utilizing this is greatly expected. For such the study, establishment of a method which effectively and assuredly selects and isolates target cells differentiated from an ES cell is one of the most important themes.

Meanwhile, when differentiated target cells express a specific membrane protein hitherto, target cells have been isolated by flow cytometry using the membrane protein as an index (Yohei Morita, Multiple Staining Analysis for Lymphocyte Subset: supervised by Hiromitsu Nakauchi, edited by Yayoi Tanaka, Freely Performed Flow Cytometry (Cell Technology Supplement), Sujunsya, p60-66(1999)/Yamashita J, Itoh H, Hirashima M, Ogawa M, Nishikawa S, Yurugi T, Naito M, Nakao K, Nishikawa S. Flk1-positive cells derived from embryonic stem cells serve as vascular progenitors. Nature 408(6808): p92-6(2000)).

However, since application of this method can be limited to the case where a cell-specific molecule is expressed extracellularly as a membrane protein, application is limited, in fact, to a part of cells and organs such as hemocyte system and vascular system.

For this reason, as strategy for many cells for which a specific membrane protein is not known such as a cardiac muscular cell, there is reported a method of stably transferring into an ES cell a recombinant gene in which a marker gene is ligated under a promoter region of a molecule (gene) expressed specifically in a target cell, and selecting and isolating a cell using a marker expressed specifically in a differentiated cell as an index (Andressen C, Stocker E, Klinz F J, Lenka N, Hescheler J, Fleischmann B, Arnhold S, Addicks K. Nestin-specific green fluorescent protein expression in embryonic stem cell-derived neural precursor cells used for transplantation. StemCells 19(5):419-24(2001)). This method is a method of using a promoter of a gene expressed specifically in a tissue to express a drug-resistant gene tissue-specifically, and selecting only a target cell using the drug, or a method of selecting and isolating a target cell by flow cytometry by expressing cell-specifically a molecule which develops a color with excitation light having a specific wavelength.

However, these methods have a great problem that they remarkably depend on activity (expression intensity) of a cell-specific promoter, and their application and efficacy are considerably limited. That is, when a cell-specific promoter has, if any, weak specificity, since sufficient intensity of expression of a marker gene for selecting and isolating a differentiated target cell can not be obtained, and a target cell can not be selected, this method has, in fact, considerably limited application. In addition, it is difficult to predict whether this method is assuredly useful or not at a stage of planning an experiment, and predicted result is not certain. Nevertheless, for differentiation into a target cell and isolation of the cell, an ES cell strain must be prepared one by one every purpose, and a differentiated target cell must be tested one by one, therefore, a target cell can not be consequently isolated in some cases although much labor and time are consumed.

By allowing even a tissue-specific promoter having low activity (expression intensity) to be expressed at a sufficient amount in order to have a marker gene function, the present invention provides a method for assuredly, simply and rapidly selecting and isolating a differentiated target cell which can be utilized in various study fields such as medicine, biology and biotechnology as well as regeneration therapy using ES cells of various animals, a method for selectively visualizing such the target cell, and a kit for isolation and visualization which is used therefor.

### DISCLOSURE OF THE INVENTION

The present invention is a method for selectively isolating or visualizing a target cell differentiated from an embryonic stem cell, which comprises transferring a first recombinant DNA in which a first promoter, a gene having recombinase-recognition sequences on both ends, and a selective marker gene for a target cell differentiated from an embryonic stem cell are arranged in this order from a 5′ side, and the first promoter makes the selective marker gene to be expressed, and a second recombinant DNA in which a second promoter specifically expressing a target cell differentiated cell embryonic stem cell, and a recombinase-expressing gene are arranged in this order from a 5′ side, into an embryonic stem cell, respectively. In the aforementioned invention, transfer of the first recombinant DNA or the second recombinant DNA into an embryonic stem cell may be performed using a vector for transferring a gene. As this vector for transferring a gene, an adenovirus vector may be used.

In addition, the present invention is an embryonic stem cell in which a first recombinant DNA and a second recombinant DNA of the aforementioned invention are transferred.

In addition, the present invention relates to a vector for transferring a first gene containing the aforementioned first recombinant DNA or a vector for transferring a second gene containing the aforementioned second recombinant DNA.

In addition, the present invention relates to a kit for isolation or visualization used for a method for selectively isolating or visualizing a target cell differentiated from an embryonic stem cell, which comprises a first vector for transferring a gene containing the aforementioned first recombinant DNA, and a second vector for transferring a gene containing the aforementioned second recombinant DNA.

In addition, the present invention relates to a kit for isolation or visualization used in a method for selectively isolating or visualizing a target cell differentiated from an embryonic cell, which comprises an embryonic stem cell in which the aforementioned first recombinant DNA is transferred, and a second vector for transferring a gene containing the aforementioned second recombinant DNA.

In addition, the present invention relates to a kit for isolation or visualization used in a method for selectively isolating or visualizing a target cell differentiated from an embryonic stem cell, which comprises a first vector for transferring a gene containing the aforementioned first recombinant DNA, and an embryonic stem cell in which the aforementioned second recombinant DNA is transferred.

In addition, the present invention relates to a cell obtained by the aforementioned method for selectively isolating a target cell differentiated from an embryonic stem cell or a tissue containing this cell.

In addition, the present invention relates to a method for treating a disease using the aforementioned cell and/or tissue.

The method for selectively isolating or visualizing a target cell differentiated from an embryonic stem cell of the present invention can be applied to ES cells derived from various animals. For example, the method can be used not only already established species such as mouse, rat, monkey and human, but also other species ES cells which will be established from now on, and is not particularly limited by an animal species and a kind of ES cells.

A standard method for handling ES cells is described in “Manual of Manipulation of Mouse Embryo” authored by Brigid Hogan and others, translated by Kazuya Yamauchi and others, Kindai Publisher (1997), or “Gene Targeting: Preparation of Mutated Mouse Using ES Cells” authored by Shinichi Aizawa, Experimental Medicine Supplement, Biomanual Series 8, Yodosha (1995).

The first recombinant DNA used in the present invention is a DNA in which a first promoter, a gene having recombinase-recognition sequences on both ends, and a selective marker gene for a target cell differentiated from an embryonic stem cell are arranged in this order from 5′, which has been made by the gene recombination technique. The first promoter is not particularly limited as far as it is a promoter which has higher activity than that of a second promoter being not capable of sufficiently expressing a selective marker gene, and is sufficient for expressing a selective marker gene. For example, a constitutive strong expression promoter such as a CA (hybrid promoter of cytomegalovirus enhancer and chicken β actin promoter) promoter and a CMV (cytomegalovirus early gene enhancer•promoter) promoter is suitable. The constitutive strong expression promoter refers to a promoter which has a target gene to be expressed constitutively and strongly when a target gene ligated to the promoter is transferred into most cells including an ES cell.

The recombinase recognition sequence is not particularly limited as far as it is a base sequence recognized by recombinase which is a specific DNA recombinant enzyme, and refers to a specific base sequence such as loxP and FRT which causes a DNA recombination reaction such as cleavage, substitution and binding of a DNA chain held by two recombinase recognition sequences by recombinase.

The recombinase-expressing gene is a gene for expressing recombinase, and representative examples include genes expressing recombinase Cre derived from bacteriophage P1 recognizing loxP (Sternberg et al., J. Mol. Boil. Vol. 150, 467-486(1981)), recombinase FLP derived from Saccharomyces cerevisiae recognizing FRT (Babineau et al., J. Biol. Chem. Vol. 260, 12313-12319 (1985)), and R derived from pSR1 plasmid of Zygosaccharomyces ruii (Matsuzaki et al., Mol. Cell. Biol. Vol. 8, 955-962 (1988)), being not limiting.

The selective marker gene is used as an index for specifically selecting a target cell expressed by a first promoter and differentiated from an ES cell after a second recombinant DNA is transferred into an ES cell, and examples thereof include an light emitting protein gene such as EGFP (Enhanced Green Fluorescent Protein) and GFP (Green Fluorescent Protein), and various drug-resistant genes, being not limited to them as far as the gene is used as a selective marker. In particular, a light emitting protein gene is more preferable because it can visualize a target cell, and selective isolation becomes easy by using flow cytometry. In addition, since a light emitting protein gene can permanently and constitutively label and visualize also a cell which has further been differentiated from a differentiated target cell when a constitutively strong expression promoter is used, it becomes possible to observe and analyze a differentiation line and a tissue line on a culturing dish, being preferable.

The second recombinant DNA used in the present invention is a DNA in which a second promoter for specifically expressing a target cell differentiated from an embryonic stem cell, and a recombinase-expressing gene are arranged in this order from 5′, which has been made by the gene recombination technique. The second promoter refers to a promoter region of a gene which is specifically expressed only in a differentiating target cell. Examples include promoters of genes such as Nkx2.5, MEF-2, GATA-4, cardiac muscle-type actin, α-cardiac myosin heavy chain (hereinafter, αMHC) protein, and myosin light chain-2v (MLC2v) protein of cardiac muscle cell, nestin of brain nerve cell, glial fibrillary acidic protein (GFAP) of brain glial cell, α-fetroprotein (AFP) of more undifferentiated hepatocyte, albumin of (mature) hepatocyte, osteocalcin of osteoblast, pancreatic and duodenal homeobox gene 1 (PDX-1) of pancreatic β cell, flt-1 of blood vessel (endothelial cell), keratin 14 (K14) of an epidermal keratin cell, and muscle creatine kinase of skeletal muscle cell. Recombinase-expressing genes ligated to these promoters express recombinase only when an ES cell is differentiated into the target cell.

For transferring the first recombinant DNA or the second recombinant DNA into an ES cell, a plasmid having a drug-resistant gene together with each recombinant DNA is transferred by a general method of molecular biology such as an electroporation method, a calcium phosphate method, a liposome method and a DAE dextran method and, thereafter, a cell is cultured in a medium with a drug added for 1 to 2 weeks, thereby, a clone of an ES cell in which each of these recombinant DNAs is incorporated into a chromosome and which expresses a gene stably and permanently is collected, and can be used in an experiment for differentiation. As a further useful method, each vector for transferring a gene containing the first recombinant DNA or the second recombinant DNA is prepared, and a recombinant DNA can be transferred into an ES cell simply and very effectively by this. Examples of a vector for transferring a gene include an adenovirus vector, a retrovirus vector, a lentivirus vector, an adeno-associated vector, and a Sendaivirus vector, as well as a cationic liposome and a HVJ liposome as a none-virus vector, being not limiting.

Then, principle of the method for selectively isolating or visualizing a target cell differentiated from an embryonic stem cell of the present invention will be explained by referring to FIG. 1 using a typical example.

In the first recombinant DNA, a first promoter (in the figure, CA promoter), a loxP sequence as a recombinase recognition sequence, and an EGFP gene as a selective marker for a target cell differentiated from an ES cell are arranged in this order from a 5′ side, a Neo gene and a poly A signal as a marker are arranged between two loxP sequences, and the poly A signal is arranged downstream of the EGFP gene. The marker arranged between recombinase recognition sequences is not limited to the Neo gene, but various marker genes can be used. The poly A signal is also not particularly limited, but various poly A signals such as a poly A signal of bovine growth hormone, and a poly A signal of rabbit β-globin can be used.

In addition, in the second recombinant DNA, a second promoter (in the figure, Nkx2.5 gene promoter or αMHC gene promoter), and recombinase Cre are arranged in this order from a 5′ side. The poly A signal is arranged downstream from the recombinase Cre.

When an ES cell in which the first recombinant DNA and the second recombinant DNA are transferred is differentiation-induced into a target cell, thereby, a second promoter is expressed, and recombinase Cre acts to excise a part held by loxP sequences, an EGFP gene is strongly expressed by a first promoter, a cardiac muscular cell as a target cell can be visualized using fluorescent light as an index, and can be selectively isolated simply and easily by flow cytometry.

### BRIEF DESCRIPTION OF THE DRAWINGS

FIG. 1 is an explanation drawing showing schematically the method for selectively isolating or visualizing a target cell differentiated from an embryonic stem cell of the present invention.

FIG. 2 is a fluorescent micrograph image showing an efficacy of gene transfer by an adenovirus vector. (a) is a fluorescent micrograph image of a mouse ES cell cultured on a feeder cell (R1 cell), (b) is a fluorescent micrograph image of a mouse ES cell cultured without a feeder cell (D3 cell), and (c) is a fluorescent micrograph image of a mouse ES cell (D3 cell) during differentiation inducement. In addition, (a) to (c) are obtained by infection with Ad.CMV-LacZ at MOI of 100, and x-gal staining, and a right upper part of each fluorescent micrograph image indicates a phase contrast micrograph image thereof.

FIG. 3 is a graph showing an efficacy of gene transfer by an adenovirus vector, and (a) is a graph showing an efficacy of gene transfer in an ES cell (R1 cell) by an adenovirus vector at each MOI, and (b) is a graph showing an efficacy of gene transfer in an ES cell (D3 cell) by an adenovirus vector at each MOI.

FIG. 4 is a fluorescent micrograph image of an ES cell which has been visualized with EGFP by the method for selectively isolating or visualizing a target cell differentiated from an embryonic stem cell of the present invention. (a) Ad.CMV-LacZ was infected as a negative control, and there is no expression of EGFP. (b) Ad.CA-LacZ was infected as a positive control, and about 60 to 70% of cells were visualized by expression of EGFP. (c) Ad.Nkx2.5-Cre was infected on day 4, this was observed on day 6, and cells appearing to be a target cell are dispersed and visualized. (d) Ad.αMHC-Cre was observed on day 13, and cells appearing to be a target cell are visualized. FIG. 5 is a chart of flow cytometry of cells which were isolated using, as an index, expression of Ad.CMV-LacZ, Ad.Nkx2.5-Cre and Ad.αMHC-Cre.

FIG. 6 is a fluorescent micrograph image of immunological cell staining of cells which were isolated using, as an index, expressionof (a) Ad.Nkx2.5-Cre, or (b) Ad.αMHC-Cre. (a) A left photograph image shows EGFP, and a central photograph image shows expression of a target protein, an upper photograph shows SMA, a lower photograph shows tropomyosin (TM), and these have been obtained by immunological fluorescent staining with a specific antibody to each of them. A right photograph image is a photograph image obtained by overlaying a left photograph image and a central photograph image, showing that EGFP and a target protein are expressed in the same cell. (b) A left photograph image shows EGFP, a central photograph image shows expression of a target protein, an upper photograph shows αMHC, a lower photograph shows actinin (all cardiac muscle cell-specific molecules), and these were obtained by immunological fluorescent staining with a specific antibody to each of them. And, a right photograph image is a photograph image obtained by overlaying a left photograph image and a central photograph image, showing that EGFP and a target protein are expressed in the same cell.

FIG. 7 shows an electrophoretic image of recombinase Cre expressed by a CA promoter which is a constitutive strong expression promoter, and a Nkx2.5 promoter and an αMHC promoter of a tissue-specific gene.

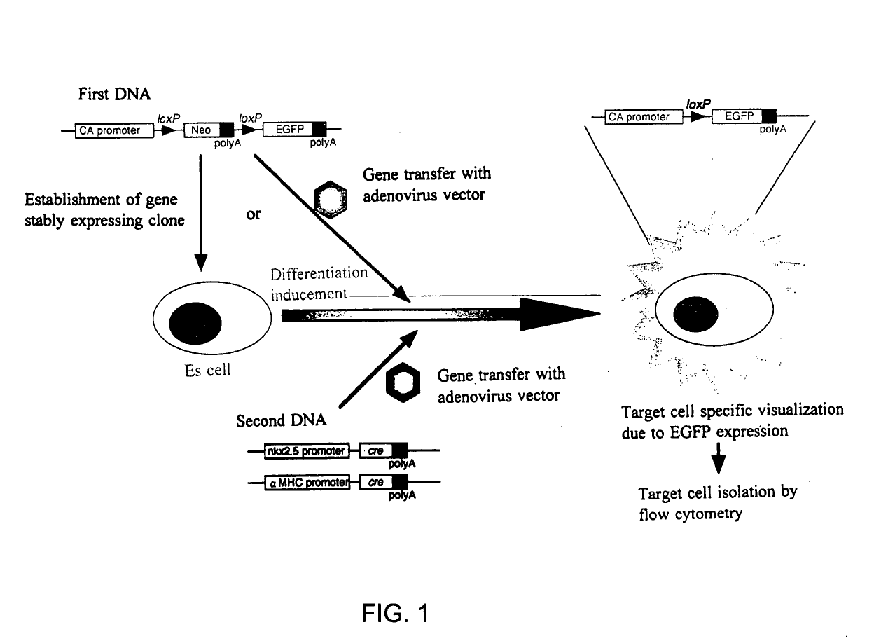

### BEST MODE FOR CARRYING OUT THE INVENTION

The present invention will be explained in detail below by way of examples. Genetic technologies and cell culturing techniques for handling plasmids, DNAs, various enzymes, Escherichia coli, and cultured cells in Comparative Examples and Examples were performed according to the methods described in “Current Protocols in Molecular Biology, edited by F. Ausubel et al., (1994), John Wiley & Sons, Inc.” and “Culture of Animal Cells; A Manual of Basic Technique, edited by R. Freshney, 2nd edition (1987), Wiley-Liss” unless otherwise indicated. In addition, culturing and handling of an ES cell were performed according to the methods described in the aforementioned “Manual of Manipulation of Mouse Embryo” authored by Brigid Hogan and others, translated by Kazuya Yamauchi and others, Kindai Publisher (1997), or “Gene Targetting: Preparation of Muted Mouse Using ES Cell” authored by Shinichi Aizawa, Experimental Medicine Supplement, Biomanual Series 8, Yodosha (1995) unless otherwise indicated. General handling of adenovirus was performed by the methods described in Manipulation of adenovirus vectors, Chapter 11. p109-p128 authored by Frank L. Graham; Methods in Molecular Biology, Vol. 7: Gene Transfer and Expression Protocols (1991) edited by E. J. Murray unless otherwise indicated. Preparation of adenovirus was performed according to the methods described in Chem, S-H. et al., Combination gene therapy for liver metastases of colon calcinoma in vivo. Proc. Natl. Acad. Sci. USA. (1995) 92, 2477-2581, or Mizuguchi et al., Human Gene Ther., Vol. 9, 2577-2583, (1998).

### COMPARATIVE EXAMPLE

The previous method for isolating an embryonic stem cell will be described below as Comparative Example.

As a Nkx2.5 gene promoter, the promoter gifted from Mr. Yutzey was used. Yutzey et al. are studying in detail a promoter region which enables regulation of cardiac muscle-specific expression of a Nkx2.5 gene by genome analysis of a 5′ upstream of the Nkx2.5 gene (details are described in Development. Vol. 125, 4461-4470 (1998)). This confirmed that a region containing a transcription initiating point to −3059 bp of 5′ upstream functions as an optimal promoter region for expression of the Nkx2.5 gene at a sufficient cardiac muscle-specific expression level.

Incidently, the aforementioned article shows that even a shorter region, that is, a region of a transcription initiating point to −959 bp of 5′ upstream, or conversely even a longer region, that is, a region of a transcription initiating point to −9000 bp of 5′ stream can not express this downstream gene at a sufficient cardiac muscle-specific expression level. A pNkx2.5-IA-LacZ plasmid in which this region of a transcription initiating point to −3059 bp of 5′ upstream of the Nkx2.5 gene, a lacZ gene of Escherichia coli as a marker gene, SV40 small t-intron and poly A signal are inserted into a plasmid pBlueScript SK was gifted from Mr. Yutzey (details are described in the aforementioned development. Vol. 125, 4461-4470 (1998)). It was confirmed that the pNkx2.5-IA-LacZ (a name described in the article is −3059Nkz2.5lacZ) expresses a LacZ gene cardiac muscle cell-specifically.

Then, an EGFP (enhanced green fluorescent protein) gene was excised from a plasmid pEGFP-C1 (Clontech, catalogue No.6084-1) by treatment with restriction enzymes NheI and BclI, and both ends of the excised EGFP gene were blunt-ended with T4 DNA polymerase I. The pNkx2.5-IA-LacZ was cut with a restriction enzyme SalI, an end was blunt-ended with T4 DNA polymerase I and, for preventing self ligation, an end was dephosphorylation-treated with a Calf Intestine Phosphatase (CIP) enzyme, and the excised EGFP gene and T4 DNA ligase were reacted to perform a ligation reaction, to prepare a plasmid pBS-Nkx2.5-EGFP.

On the other hand, a plasmid pBS-loxP-Neo was prepared by inserting a gene in which a pGK promoter, a Neo gene (G418 drug-resistant gene), and a poly A signal of bovine growth hormone were ligated in this order from a 5′ side, between HindIII-BamHI in a multicloning site between two loxP sequences of a recombinase recognition sequence of a plasmid pBS246 (formally GIBCO BRL, currently Invitrogen, catalogue No.10348-019). This plasmid pBS-loxP-Neo was cut with a restriction enzyme NotI, CIP-treated and purified to obtain a fragment, pBS-Nkx2.5-EGFP was cut with NotI to obtain a gene fragment (gene in which Nkx2.5 promoter of a transcription initiating point to −3059, an EGFP gene, SV40 small t-intron and poly A signal are ligated), and the both fragments were reacted with a T4 DNA ligase to prepare pNkx2.5-EGFP-loxP-Neo. The pNkx2.5-EGFP-loxP-Neo is a plasmid having a backbone of pBluescript, in which a Nkx2.5 gene promoter (from transcription initiating point to −3059), an EGFP gene, SV40 small t-intron and a poly A signal, a loxP sequence, a pGK promoter, a Neo gene, a poly A signal of bovine growth hormone, and a loxP sequence are inserted in this order from a 5′ side.

This plasmid pNkx2.5-EGFP-loxP-Neo was transferred into a R1 cell of a mouse ES cell strain by an electroporation method (electricity was passed at 150 mV and 950 μF with Gene Pulser II of Biorad in a 0.2 mm cuvette), G418 as an antibiotic for drug selection was added at a concentration of 150 μg/ml to an ES cell medium with LIF (recombinant protein of mouse leukemia inhibitory factor: formally GIBCO BRL, currently Invitrogen, trade name ESGRO) added from the following day and, after 1 to 2 weeks at which a colony could be sufficiently separated, this drug-resistant ES cell strain clone was collected. By three times experiments 78 G418-resistant clones were collected.

The ES cell medium is a medium in which NaHCO₃, 125 μM 2-mercaptoethanol (Nacalai), none-essential amino acid (Invitrogen), nucleic acid, 20% fetal bovine serum (Invitrogen), streptomycin and penicillin are added to Dulbecco's Modified Eagle's Medium (high glucose condition, L-glutamine and 110 mg/L sodium pyruvate are contained: Sigma). Upon maintenance of undifferentiation ability of an ES cell, culturing is performed by adding 10³ U/mL of LIF to this ES cell medium. Upon differentiation inducement of an ES cell, culturing is performed in this ES cell medium alone without adding LIF.

A DNA was extracted from these clone ES cells, genomic PCR was performed, and 25 clones were positive with primers designed for amplifying EGFP, 16 clones were positive with primers designed for amplifying a region containing a ligated Nkx2.5 gene promoter and EGFP, and 13 clones were positive with a set of both primers. Since at least these 13 clones were thought that a target gene is correctly transferred, these 13 double positive clones were used in an experiment thereafter.

ES cells of the 13 clones were placed on two differentiation inducement systems. In one system, an ES cell was cultured by suspending in an ES cell medium with LIF not added in a cell none-adhesive dish, thereby, a cell mass similar to an initial embryo called embryoid body was formed. When this embryoid body was transferred to an adhesive culturing dish from 3 day (3 days after initiation of differentiation inducement without LIF), and cultured in the adhered state in an ES cell medium with LIF not added, a cell mass which was self-pulsing like a cardiac muscle cell of a living body appeared from 7 day (7 days after initiation of differentiation inducement without LIF) to 14 day.

Another system is a differentiation inducement system in which an ES cell is placed on a mouse stromal cell called ST2 cell, and co-cultured in an ES cell medium with LIF not added. Also in this system, when an ES cell was placed on this ST2 cell without LIF and, 7 to 9 days after, a cell mass which was self-shrinking like a cardiac muscle of a living body appeared somewhere.

When a RNA was extracted from a cell mass everyday from 7 to 14 days after placement on the two differentiation systems, and a RT-PCR method was performed, expression inducement of a gene like a cardiac muscle-specific gene (Nkx2.5, αMHC, MLC2v etc.) was confirmed. In addition, expression of a muscle-specific or cardiac muscle-specific protein (α actinin, tropomyosin, Nkx2.5, MLC2v) could be confirmed by immunological staining of a cell mass at the same stage, and it was demonstrated that these self-shrinking cell masses were differentiated into a cardiac muscle cell.

On the other hand, even when a mRNA of an EGFP gene was amplified from a RNA extracted from a cell mass which had been differentiated into a cardiac muscle at 4 days after to 14 days after by a RT-PCR method, positive finding could not be seen in any case. And, in cardiac muscle cell masses differentiated from these ES cells which had been induced from any ES cell clone, neither visualization with a fluorescent microscope, nor clear expression of EGFP at a isolatable level with a cell sorter could not be seen. As described above, this Nkx2.5 gene promoter (−3059) is the same as that reported by Yutzey et al. in Development. Vol. 125, 4461-4470 (1998), a gene ligated to this promoter was expressed at a region which is scheduled to be a heart at an early stage in development of a mouse, and it was confirmed that the gene is expressed in a cardiac muscle cell relatively specifically.

Since the method for detecting expression of a LacZ gene by x-gal staining of a tissue used by Yutzey et al. is an enzymatic reaction, detection sensitivity is relatively high, and a level of expression of a LacZ gene necessary for detection may be very low, but in order to visualize or isolate a cell while living, a LacZ gene cannot be used as a marker gene. For this reason, it is general to use an EGFP gene as a marker gene for such the purpose. On the other hand, in order to visualize EGFP with a fluorescent microscope or separate it with a cell sorter, an extent of an expression amount of an EGFP gene is necessary. That is, although this gene has been correctly incorporated, and an ES cell has been differentiated into a cardiac muscle, expression of EGFP is not seen because a gene ligated to this Nkx2.5 gene promoter produces cardiac muscle-specific expression, but has not expression activity at a level at which EGFP can be visualized. Like this, by the previous method, a cardiac muscle cell which had been differentiated into a target cell could not be visualized by expression of an EGFP gene from a Nkx2.5 gene promoter, and could not be isolated with a cell sorter, thus, both trials were impossible.

### Example 1

Both of pCMV-loxP-Neo-EGFP and pCA-loxP-Neo-EGFP, a plasmid containing a first recombinant DNA in which a CMV promoter (human cytomegalovirus immediate early promote) or a CA promoter (cytomegalovirus enhancer+chicken β-actin promoter), a Neo gene held by two loxP sequences downstream therefrom, and an EGFP gene as a marker gene further downstream therefrom were arranged from a 5′ side, were prepared, and the preparation process will be described below.

First, a pcDNA3 plasmid (Invitrogen) is treated with restriction enzymes BclI and BsmI, a Neo gene is excised, and the fragment was blunt-ended by T4 DNA polymerase I treatment (insert). A pBS246 plasmid (formally GIBCO BRL, currently Invitrogen) is cut with restriction enzymes HindIII and BamHI, and CIP-treated (vector). Both of this insert and the vector were reacted with a T4 DNA ligase to perform ligation, to obtain a plasmid pBS-loxP-Neo in which a Neo gene was inserted between loxP sequences of pBS246.

Then, this pBS-loxP-Neo was excised with a restriction enzyme NotI, and treated with T4 DNA polymerase I to recover two loxP sequences and a gene fragment of a Neo gene held by them (insert). On the other hand, a plasmid pIRES-EGFP (Clontech, catalogue No.6064-7) with a CMV promoter transferred therein was treated with restriction enzymes ClaI and BamHI, a vector part from which MCS (multicloning site), IVS (synthetic intron), and IRES (internal ribosome entry site) had been removed was recovered, and this was blunt-ended with T4 DNA polymerase I (vector). Both of this insert and the vector were ligated with a T4 DNA ligase, to obtain one target plasmidp CMV-loxP-Neo-EGFP.

Further, the plasmid pCMV-loxP-Neo-EGFP was treated with BglII and EcoRI to remove a CMV promoter part, the fragment was blunt-ended with T4 DNA polymerase I, and CIP-treated (vector). On the other hand, a cosmid pAdexlCawt (Takara, code No.6150) was treated with PmeI and SwaI to excise a CA promoter part, and the fragment was blunt-ended with T4 DNA polymerase I (insert) Both of this insert and the vector were reacted with a T4 DNA ligase to perform ligation, to obtain another end plasmid pCA-loxP-Neo-EGFP.

The plasmid pCMV-loxP-Neo-EGFP or pCA-loxP-Neo-EGFP was transferred into an ES cell (D3 cell), respectively, by an electroporation method, and this was cultured for 1 to 2 weeks in an ES cell medium with 150 μg/mL G418 and 10³U/L LIF added thereto, to isolate an ES cell clone. These procedures are as described in detail in item of Comparative Example. Then, 70 clones in which pCMV-loxP-Neo-EGFP was transferred, and 59 clones in which pCA-loxP-Neo-EGFP was transferred, were collected.

Separately, a second recombinant DNA in which a Nkx2.5 promoter (−3059), a recombinase Cre gene, and a bovine growth hormone poly A signal were arranged from a 5′ side was prepared, and an adenovirus vector Ad.Nkx2.5-Cre containing the gene was further prepared as follows:

First, a plasmid pHMCMV6 (gifted from Mr. Mark Kay: details of the plasmid are described in H. Mizuguchi and M. Kay: Human Gene Ther vol. 10 2013-201 (1999); currently sold by Clontech) was treated with NheI and MunI to remove a CMV promoter, and ligation was performed with a T4 DNA ligase. Further, this plasmid was cut with AflII, the fragment was blunt-ended with T4 DNA polymerase I (vector), and an insert obtained by excising a Cre gene with XhoI and MluI from a plasmid pBS185 (formally GIBCO BRL, currently Invitrogen), and blunt-ending this with T4 DNA polymerase I was inserted in this place by a ligation reaction with a T4 DNA ligase to obtain a plasmid pHM Ap-Cre. That is, pHM Ap-Cre is a plasmid which does not have a promoter, has a multicloning site for simply inserting an arbitrary promoter, and has a Cre gene, and a bovine growth hormone poly A signal downstream therefrom.

The pNXK2.5-IA-LacZ described in Comparative Example was excised with NotI and XbaI, and this was blunt-ended with a T4 DNA polymerase to obtain a Nkx2.5 gene promoter part (insert), which was ligated to a vector obtained by cutting pHM Ap-Cre with NotI, blunt-ending with T4 DNA polymerase I, and CIP-treating it, with a T4 DNA ligase, to obtain a plasmid pHM-Nkx2.5-Cre.

Further, an insert obtained by cutting pHM-Nkx2.5-Cre with I-CeuI and PI-SceI, and a vector obtained by cutting an adenovirus vector plasmid pAdHM4 (gifted from Mr. Mark Kay: details of the plasmid are described in H. Mizuguchi and M. Kay: Human Gene Ther vol. 10: 2013-201 (1999)) with I-CeuI and PI-ScuI were ligated with a T4 DNA ligase to obtain an adenovirus vector plasmid pAdHM4-Nkx2.5-Cre. The pAdHM4-Nkx2.5-Cre was cut with PacI and purified, which was transferred into a 293 cell, a plaque of adenovirus Ad.Nkx2.5-Cre appearing after 10 to 14 days was recovered, amplification of the virus was performed with a 293 cell, purified by a density gradient method with CsCl, and subjected to desulting with a column (details of the method are described in the cited reference described first).

After infection of a cell with a human 5-type adenovirus vector and transfer into the cell, this Ad.Nkx2.5-Cre expresses a Cre gene under control of a Nkx2.5 gene promoter.

In addition, according to a similar method, a CA promoter was transferred into pHMAp-Cre and, according to a similar method, an adenovirus vector Ad.CA-Cre was prepared. The Ad.CA-Cre is a human 5-type adenovirus vector and, after infection of a cell and transfer of a gene into the cell, the vector expresses a Cre gene under control of a CA promoter. Since the CA promoter can constitutively and strongly express a downstream gene in most cells including an ES cell, a cell infected with Ad.CA-Cre expresses constitutively a Cre enzyme in the cell although in the undifferentiated state.

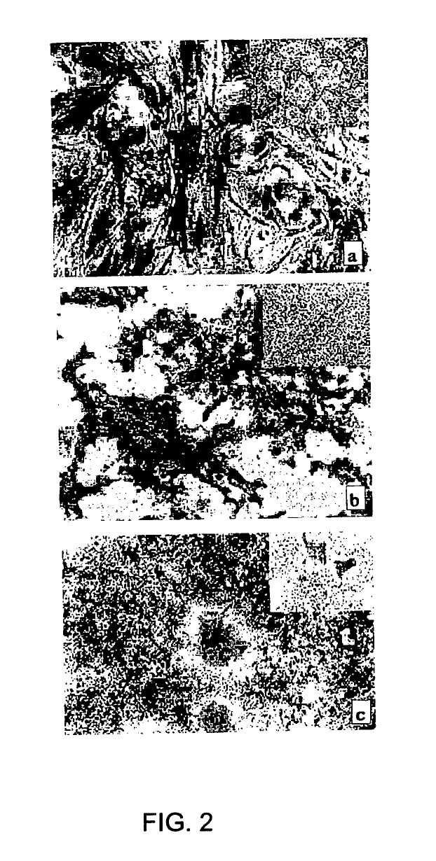

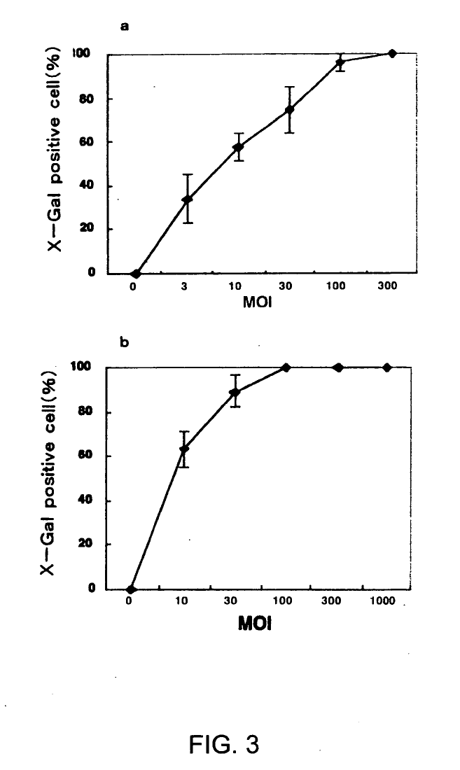

Then, an efficacy of transfer of a gene into an ES cell of an adenovirus vector was investigated. First, a human 5-type adenovirus vector Ad.CMV-lecZ expressing a LacZ gene under control of a CMV promoter was prepared by the aforementioned method. When a cell was infected with the Ad.CMV-LacZ at each MOI (multiplicity of infection: infectable virus number/cell number), and a ratio of positive cells was assessed by x-gal staining a gene transfer efficacy was increased as MOI was increased (see FIG. 2, FIG. 3). And, in an ES cell of any of R1 and D3, a gene could be transferred in many cells at MOI of 30 in the presence or in the absence of a feeder cell, and a gene could be transferred into 100% of ES cells at MOI of 100 to 300. However, since cell disorder is seen in some cases when MOI is too extremely increased, an experiment described later was performed at MOI of 30 at which a gene can be transferred into about 60 to 80% of cells while little cell disorder is seen. Similarly, when an ES cell which had been subjected to the aforementioned differentiation inducement was infected with a sufficient amount of Ad.CMV-LacZ, and this was assessed by x-gal staining, a gene could be transferred into almost cells with a slight difference at any differentiation stage and any day of 1 to 14 days after differentiation inducement (see FIG. 2c) It could be confirmed that a gene can be transferred into an ES cell simply and at a high rate at any differentiation stage by using an adenovirus vector like this. In particular, since it is difficult to transfer a gene by the previous general gene transferring method such as an electroporation method during differentiation inducement, it was seen that use of an adenovirus vector is very useful.

Among 70 clones of an ES cell in which pCMV-loxP-Neo-EGFP with a first recombinant DNA incorporated therein was stably transferred, first, 34 was subjected to genomic PCR with a primer set designed in Comparative Example for amplifying EGFP, and 31 among them were positive. In addition, when these 31 ES cell clones were infected with Ad.CA-Cre, 4 clones were visualized with an expression intensity sufficient for observation with a fluorescent microscope (all of these four clones are positive by genomic PCR with EGFP). That is, since a clone having more intense expression can be rapidly selected by direct screening with Ad.CA-Cre infection even when insertion of an EGFP gene is not confirmed by genomic PCR, the resulting ES cell clone was selected thereafter for a target clone by expression intensity of EGFP by direct Ad.CA-Cre infection. When remaining 34 clones with pCMV-loxP-Neo-EGFP transferred stably were screened by EGFP expression intensity (by observation with a fluorescent microscope) after Ad.CA-Cre infection, 11 clones were EGFP strong expression, and 4 clones were EGFP weak expression. That is, target 15 clones (19 clones including weak expression) could be collected from a total of 70 clones with pCMV-loxP-Neo-EGFP transferred stably.

On the other hand, when 59 clones of an ES cell with pCA-loxP-Neo-EGFP transferred stably were directly screened by EGFP expression after Ad.CA-Cre infection, 16 clones were target clones expressing EGFP strongly.

In both of an ES cell with pCMV-loxP-Neo-EGFP transferred stably and an ES cell with pCA-loxP-Neo-EGFP transferred stably, expression of EGFP at a visible level was not seen, and problematic background was not seen when not infected with Ad.CA-Cre, or when infected with Ad.CMV-LacZ as a control not expressing a Cre gene. In addition, an expression level of EGFP of an ES cell with each of PCMV-loxP-Neo-EGFP and pCA-loxP-Neo-EGFP transferred stably after Ad.CA-Cre infection was stronger in pCA-loxP-Neo-EGFP in any case. This seems to be due to a difference in promoter activities that a CA promoter can induce stronger expression than a CMV promoter also an ES cell as reported in many cell strains. However, expression of EGFP at a visible level was obtained also in an ES cell with pCMV-loxP-Neo-EGFP, and it is considered that even pCMV-loxP-Neo-EGFP has essentially no problem in an experiment for the purpose of analysis and isolation with a cell sorter having good sensitivity. The following differentiation inducement experiment in the present experimental example was performed using pCA-loxP-Neo-EGFP from a viewpoint that expression is stronger, observation under a fluorescent microscope can be performed more clearly, and analysis can be performed more easily.

Among 15 clones of an ES cell for which strong expression of EGFP is assured by stable transfer of pCA-loxP-Neo-EGFP and excision of a Neo gene held by loxPs after expression of a Cre enzyme as described above, 3 clones were used to perform a differentiation inducement experiment using Ad.Nkx2.5-Cre. In a preliminary experiment using the aforementioned Ad.CA-Cre, expression of EGFP at a sufficiently visible level was seen from one day after Ad.CA-Cre infection, expression reached a maximum expression level two days after infection, and it was confirmed that sustained expression of EGFP at the same level is obtained thereafter.

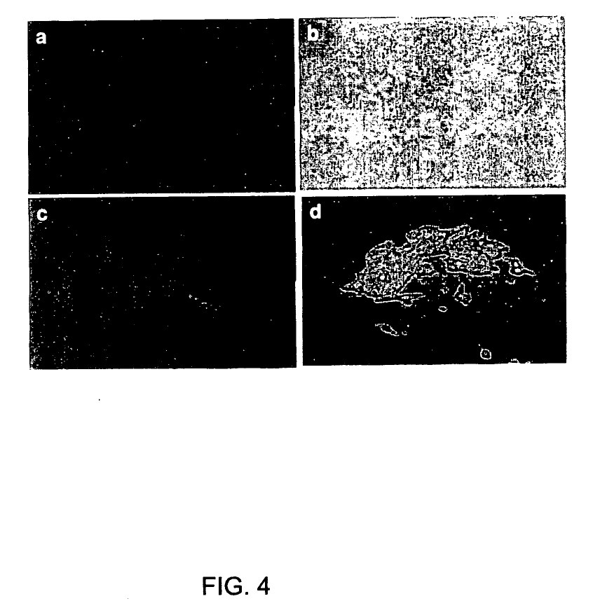

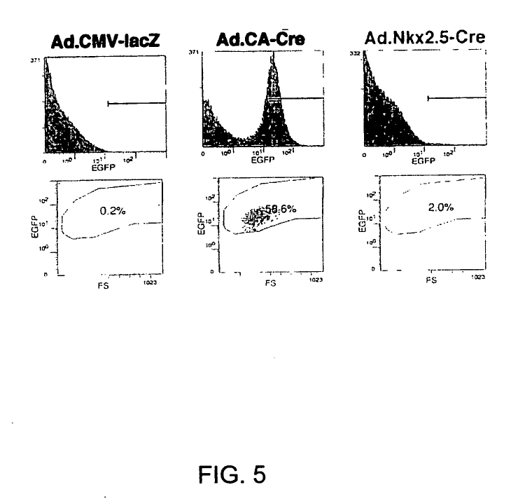

These 3 clones of an ES cell were used in the differentiation inducement system via embryoid body already described in Comparative Example. First, expression of a Nkx2.5 mRNA in this differentiation inducement system was examined by RT-PCR everyday, and expression of Nkx2.5 was clearly seen from 5 days after differentiation inducement. For this reason, 4 days after differentiation inducement an ES cell during differentiation was infected with Ad.Nkx2.5-Cre at MOI of 30. That is, on day 1, the cell was cultured in an ES cell medium with LIF not added, preparation of embryoid body was initiated in the none-adhered state, this was adhered on a culturing dish 3 days after initiation of differentiation inducement and, on day 4, the cell was infected with Ad.Nkx2.5-Cre at MOI of 30. From 5 days after initiation of differentiation inducement, cells appeared somewhere for which EGFP could be visualized under a fluorescent microscope, from 6 days after initiation of differentiation inducement, EGFP expression at a very strong expression level was observed under a fluorescent microscope, and this EGFP expression was ever seen thereafter (see FIG. 4C). On day 6 or day 8, a cell mass was dissociated with trypsin, and sufficiently separated into individual cells, and a target cell expressing EGFP was isolated with a cell sorter. By analysis with this flow cytometry, a positive rate of a group infected with Ad.Nkx2.5-Cre, that is, a Nkx2.5-expressing cell was about 2% (see FIG. 5). To the contrary, a positive rate of a group infected with Ad.CA-Cre instead of Ad.Nkx2.5-Cre as a positive control (that is, this represents a ratio of a cell which was infected with adenovirus and in which a Cre gene was transferred) was about 60% (see FIG. 5). On the other hand, a positive ratio of a group infected with Ad.CMV-LacZ instead of Ad.Nkx2.5-Cre as a negative control, or a group not infected with an adenovirus vector at all (that is, this is leakage of EGFP expression in an ES cell with a gene of pCA-loxP-Neo-EGFP transferred stably in the state where loxPs have not been excised with a Cre enzyme, background) was 0 to 0.2% (see FIG. 4a, FIG. 5).

That is, in this experimental system, since a target gene is correctly transferred in about 60% of cells at a very high rate, there is no background, and a target Nkx2.5-expressing cell can be visualized and isolated correctly, it was seen that the experimental system is an very excellent experimental system which has solved these techniques which could not be solved previously.

Then, the isolated cell was immediately seeded on culturing a cell, and cultured and, on the following day, or a few days after, nature and character of the cell were analyzed. First, the isolated cell exhibited strong EGFP expression to an extent that expression could be clearly confirmed in most cells with a fluorescent microscope from on the following day to a few days after. Thereby, it was confirmed that the cell isolated by this method is a target cell having a very high purity. Then, for analyzing nature and character of the cell, a RNA was extracted from this cell, and expression of a Nkx2.5 gene, and expression of a cardiac muscle-specific molecule, and other associated genes were investigated. Alternatively, these isolated cells were subjected to immunological cell staining, and expression of these molecules was investigated.

First, clear expression of a Nkx2.5 gene, αMHC, MEF2c (cardiac muscle-specific transcription factor) or GATA4 (transcription factor expressed in a cardiac muscle at highly frequently) was seen in 80% of cells. This result revealed that most of these cells are a cardiac muscle cell.

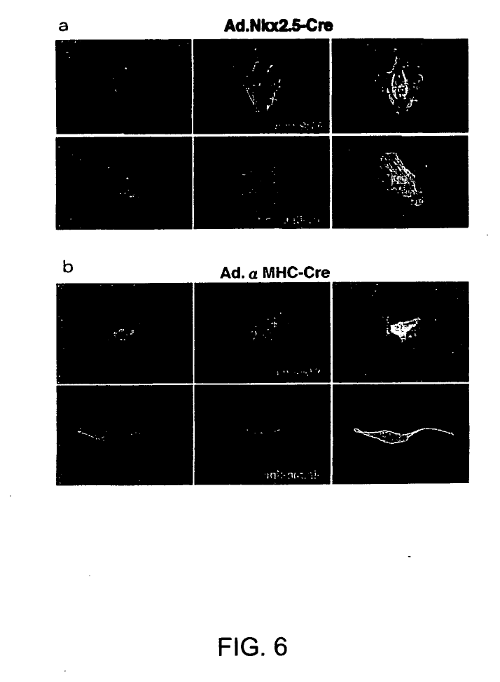

On the other hand, expression of smooth muscle actin (SMA) or tropomyosin (TM) which is a marker for other than a cardiac muscle was also seen in 20 to 30% of cells (see FIG. 6a). There has been previously no report that only a cell expressing Nkx2.5 could be isolated assuredly (moreover, assuredly at such a level that it can be also visualized with a fluorescent microscope) from an ES cell, and when taken into consideration that Nkx2.5 is a transcription factor which is expressed earliest during a process of cardiac muscle development among cardiac muscle-specific genes known today, result that a part of the present cell expresses a marker for other than a cardiac muscle may lead to a possibility that the present cell is an more undifferentiated cell which has not previously isolated from an ES cell and is destined to be differentiated into a cardiac muscle cell at a differentiation stage before a mature cardiac muscle cell, or further a cardiac muscle which should be called a cardiac myoblast. For this, this isolated cell itself is considered to be very useful particularly in future fundamental study regarding development, differentiation or regeneration of a heart at an early stage which has not been sufficiently clarified yet, or development of regeneration therapy in a cardiac disease using an ES cell.

In addition, as various cardiac muscle-specific genes expressed at each differentiation stage of a cardiac muscle, some such as MEF2C and GATA4 have been reported and, from now on, a cell at each differentiation stage can be freely isolated by using the present method and using expression of such the gene as an index, thus, the invention of the present method is greatly meaningful. Further, usefulness of the present method is not limited to a cardiac muscle, but by using promoters of tissue-specific or character-identified various genes, each tissue at any specific differentiation stage differentiated from an ES cell, or any target cell specialized for expression of a certain gene can be visualized and isolated. Also from this point of view, the present invention has extremely important meaningfulness in regeneration medicine or embryology using an ES cell.

### Example 2

The second promoter used in the present invention is not limited to the Nkx2.5 gene promoter, but can be widely used by generalization for the purpose of visualizing and further isolating an ES cell-derived cardiac cell or ES cell-derived other cells or tissues using other cardiac muscle-specific gene as an index under fluorescent microscope. That is, only by substituting the second promoter with an objective promoter, any ES cell-derived target cell can be visualized and isolated. In order to further confirm such the general wide usefulness of the present invention, an adenovirus vector Ad.αMHC-Cre using a mouse AMHC gene promoter as the second promoter, that is, an adenovirus vector for transferring a recombinant gene specifically expressing a Cre enzyme into a cardiac muscle cell in which AMHC is expressed, was prepared by the same method as that of Example 1, and the same experiment was performed.

The Ad.αMHC-Cre was prepared as follows: First, about 5.5 kb of a DNA containing an αMHC gene promoter was excised from a plasmid with an αMHC promoter inserted therein (details of the plasmid are described in J. Biol. Chem. Vol. 266, p9180-9185(1991)) which had been gifted from Mr. Jeffrey Robbins of University of Cincinnati, College of Medicine, blunt-ended with T4 DNA polymerase I, purified, and extracted. As Mr. Robbins et al. reported in the aforementioned Publication, this is a DNA containing a part of a 3′ last exon of a heart-type β myosin heavy chain gene, a 3′ region of AMHC, and first three exons not encoding a protein, and the same article shows that this region functions as a promoter resulting in heart-specific expression. On the other hand, the vector pHM Ap-Cre prepared in Example 1 was cut with an enzyme NotI, blunt-ended with T4 DNA polymerase I, subjected to terminal dephosphorylation treatment with an enzyme CIP, and purified. This and a 5.5 kb DNA fragment of the aforementioned αMHC gene promoter were reacted with a T4 DNA ligase enzyme to perform a ligation reaction, to prepare a plasmid pHM-α MHC-Cre in which a Cre gene is ligated to downstream of the αMHC gene promoter. This pHM-αMHC-Cre and the adenovirus vector plasmid pAdHM4 were cut with restriction enzymes I-CeuI and PI-SceI as described in Example 1, and both plasmids were ligated with a T4 DNA ligase to obtain pAdHM4-αMHC-Cre. The pAdHM4-αMHC-Cre was cut and purified, which was transferred in a 293 cell, a plaque of the adenovirus Ad.αMHC-Cre appearing 10 to 14 days after was recovered, and the virus was amplified, purified, and desalted as described in Example 1. The thus prepared Ad.αMHC-Cre expresses a Cre gene under control of the AMHC gene promoter after infection of a cell and transfer of a gene into the cell.

Among the ES cell clone strains in which the pCA-loxP-Neo-EGFP prepared Example 1 was stably transferred, the ES cell clone strain for which strong expression of EGFP after expression of Cre was confirmed was used to perform the same experiment as that of Example 1 employing Ad.αMHC-Cre in place of Ad.Nkh2.5-Cre. The fundamental experimental protocol and procedure were the same as those described in Example 1, provided that in Example 1, a cell was infected with Ad.Nkx2.5-Cre 4 days after differentiation inducement, and the visualized target cell was isolated with a cell sorter on day 6, while in the present Example, a cell was infected with Ad.1αMHC-Cre on day 9, and a visualized target cell was isolated with a cell sorter on day 13. The reason is as follows: When expression of an endgenous αMHC gene in an ES cell after initiation of differentiation inducement was investigated by a RT-PCR method and immunological histochemistry, expression of αMHC was remarkably perceived from about day 8 to day 14. The result of a time of expression of this αMHC in differentiation of an ES cell is consistent with the fact in a living body that since αMHC is one of cardiac muscle-specific shrinkage proteins, strong expression is recognized in a mature cardiac muscle cell, and is also consistent with the fact that a colony of an ES cell exhibiting cardiac muscle-like pulsing is most remarkably recognized on day 9 to 14 after differentiation inducement. Like this, according to the same manner as that of Example 1 except that a time of infection with Ad.αMHC-Cre, the following experiment was performed.

From day 1 after infection with Ad.αMHC-Cre (day 9 after differentiation inducement), expression of EGFP was recognized in a part of cells under a fluorescent microscope, from day 2 after infection (day 10 after differentiation inducement), expression became clear, thereafter, expression was slightly enhanced and, on day 4 after infection (day 13 after differentiation inducement), expression of EGFP became maximum. Therefore, at this term point, cells were dissociated with trypsin, and EGFP-positive cells were isolated with a cell sorter (see FIG. 4d). Most of the isolated cells were expressing EGFP. Further, in order to confirm property of these cells, a mRNA of a cardiac muscle-specific molecule such as αMHC and actinin, and expression of a protein were investigated by RT-PCR and immunological staining, and expression of these cardiac muscle-specific molecules was positive in most of the isolated cells (see FIG. 6b). Further, a cell structure peculiar in a cardiac muscle cell such as a striated muscle fiber structure could be confirmed by observation with an electron microscope. Inter alia, observation of cardiac muscle-like pulsing of a cell for a few days from the next day after culturing while a cell was contacted with a culturing dish showed that the isolated cell is a target matured cardiac muscle cell. From these results, it was confirmed that the isolated cell is a matured cardiac muscle cell. Like this, since a cardiac muscle cell at a relatively unmatured differentiation stage could be assuredly isolated in Example 1, and a matured cardiac muscle cell could be assuredly isolated in Example 2 using promoters of two genes having such the different characters that Nkx2.5 is a transcription factor and αMHC is a shrinkage protein, by the present method, it was confirmed that the present invention is widely used by generalization, and is useful.

### Example 3

While a method of taking first an ES cell clone in which the first recombinant DNA could be stably transferred, and transferring the second recombinant DNA into the ES cell by an adenovirus vector during a process of differentiation inducement was used in Example 1, the first recombinant DNA and the second recombinant DNA were directly transferred in an ES cell using an adenovirus vector during a process of differentiation inducement, without the work of taking an ES cell clone in Example 3.

For doing so, first, an adenovirus vector into which the first recombinant DNA can be transferred, was prepared as follows:

First, a target DNA fragment in which a CA promoter, a neo gene held by loxP sequences of a recombinase recognition sequence, a poly A sequence, an EGFP gene, and a poly A sequence were ligated from a 5′ side was excised from the pCA-loxP-Neo-EGFP and the pCMV-loxP-Neo-EGFP prepared in Example 1 by SalI enzyme treatment. On the other hand, a SalI recognition sequence of a multicloning site of a pHM5 plasmid (gifted from Mr. Mark Kay: details of the plasmid are described in H. Mizuguchi and M. Kay. Human Gene Ther vol. 10:2013-2017(1999); having restriction enzyme recognition sequences of 1-CeuI and PI-SceI, on both ends of a multicloning site, respectively) of a shuttle vector for preparing adenovirus was cut with a SalI enzyme, subjected to terminal dephosphorylation treatment with a CIP enzyme, and this and the aforementioned excised target DNA fragment were ligated with a T4 DNA ligase enzyme to obtain shuttle vector plasmids pHM-CA-loxP-Neo-EGFP and pHM-pCMV loxP-Neo-EGFP in which the objective first recombinant DNA was inserted, respectively. Further, a target gene part was excised from the pHM-CA-loxP-Neo-EGFP or the pHM-pCMV-loxP-Neo-EGFP with an enzyme I-CeuI or PI-SceI, and an adenovirus vector plasmid pAdHM4 treated with enzymes-CeuI and PI-SceI was ligated with a T4 DNA ligase as also described in Example 1 to obtain adenovirus vector plasmids pAdHM4-CA-loxP-Neo-EGFP and pAdFM4-pCMV-loxP-Neo-EGFP having the objective first recombinant DNA, respectively.

Preparation of an adenovirus vector was performed as described in Example 1. That is, the pAdHM4-CA-loxP-Neo-EGFP or the pHdHM4-pCMV-loxP-Neo-EGFP was treated with a PacI enzyme, transfected into a 293 cell, and the resulting virus plaque was amplified, purified and desalted to obtain an adenovirus vector Ad.CA-loxP-Neo-EGFP or Ad.CMV-loxP-Neo-EGFP containing the objective first recombinant DNA.

As described also in Example 1, there is no essential difference in a CA promoter and a CMV promoter in an object of the present Example, but results of an experiment using Ad.CA-loxP-Neo-EGFP are shown below due to stronger expression. In this respect, the same experiment was performed using Ad.CMV-loxP-Neo-EGFP, and it was confirmed that the same results are obtained.

Using an ES cell with no gene transferred (D3), differentiation inducement was performed by preparing embryoid body in a medium excluding LIF as in Example 1.

First, for isolating an more undifferentiated cardiac muscle cell derived from an ES cell using Nkx2.5 as an index, two adenovirus vectors of Ad.Nkx2.5-Cre and Ad.CA-loxP-Neo-EGFP were infected at MOI of 30 on day 4, and a target EGFP-expressing cell was isolated with a cell sorter on day 6. That is, the present Example is different from Example 1 only in that the first recombinant DNA was transferred using an adenovirus vector. As a result of this experiment, the same cells as those of Example 1 were isolated, and these cells exhibited the same gene expression pattern as that of Example 1.

Then, for isolating a mature cardiac muscle cell derived from an ES cell expressing an αMHC gene, two adenovirus vectors of Ad.αMHC-Cre and Ad.CA-loxP-Neo-EGFP were infected at MOI of 30, respectively, on day 9 after differentiation inducement as in Example 2, and a target EGFP-expressing cell was isolated with a cell sorter on day 13. This experimental result also showed that the same cells as those of the experiment 2 were isolated, and these cells exhibited a gene expression pattern and cardiac muscle cell-like shrinkage characteristic in a cardiac muscle similarly.

From results of these two experiments, it was confirmed that, by transferring the first recombinant DNA or the second recombinant DNA using adenovirus, the same result as that of the previous method of taking a stably expressing cell with a gene transferred therein can be obtained more simply.

A method of preparing an ES cell stably expressing the first recombinant DNA by the prior art, selecting a better clone, and transferring the second recombinant DNA into the clone with adenovirus, as shown in Example 1 and Example 2, and a method of transferring both DNAs using adenovirus as in Example 3 have no essential difference in meaningfulness in the present invention and, on the other hand, it is thought that it is useful to use respective advantages of the two methods depending on an object.

Advantages of using an adenovirus vector for transferring two DNAs are summarized as follows:

(1) The first and second recombinant DNAs are stably transferred, and labor and a time of troublesome work of taking a constitutively expressing clone are not necessary: That is, as shown in Examples 1 and 2, in order to transfer the first recombinant DNA by the prior art, and selectively take a stably and constitutively expressing clone, labor and a time are necessary. Further, if transfer of a gene is performed only by the prior art without using no adenovirus vector, and a clone stably and constitutively expressing both of the first and second recombinant DNAs is selectively taken, a further time and further labor are necessary. In this case, besides, two kinds of different drug-resistant genes are necessary, there is a possibility that troublesome work of expression of these multiple drug-resistant genes, and selection of the clone, and influence of long term drug use on an Es cell, that is, change in character of a cell become problematic, and a target clone can not be taken due to such the various influence in some cases.

(2) An adenovirus vector can transfer a target gene simply and at a high rate at an arbitrary time of a differentiation stage: This was impossible by the prior art, but the present invention has shown that this can be simply done using adenovirus. Advantage from this is associated with the description of (1) and is that unnecessary influence such as unnecessary long term exposure to a gene and a drug is not given to an ES cell.

(3) Since an adenovirus vector stably expresses a gene transferred into a host cell (in this case, ES cell) as an episomal form (presence in a nucleus without incorporation into a chromosome) for a long term, stable result is obtained: In the case of a method of taking an ES cell clone in which a target gene is incorporated into a chromosome utilizing a drug-resistant gene by the previous method, since the transferred gene is randomly incorporated into a chromosome, there is influence of a place where a gene is incorporated on a chromosome, or influence of a chromatin structure. For this reason, since all incorporated genes are not necessarily expressed stably, a time and labor are necessary for selecting a better clone stably expressing an transferred gene as described in (1). Further, in the case of an ES cell, it is known that expression of a gene incorporated into a chromosome easily becomes unstable, for example, expression of an transferred gene is shut off in some cases, as compared with the case using other cancer cell strain or a primary normal cultured cell. To the contrary, when a gene is transferred with an adenovirus vector, there is hardly such the influence of a chromosome or chromatin due to episomal, stable expression is obtained, and reproducible and stable result is always obtained.

(4) An arbitrary ES cell strain can be used: Differentiating ability and character are different depending on a kind of an ES cell strain, and a clone and a subclone thereof. In this case, for isolating a certain cell, there is contemplated the case where a clone of an ES cell strain which has been identified that the clone is more easily differentiated into the cell is intended to be particularly used. By using an adenovirus vector which can transfer the first and second recombinant DNAs of Example 3, it is not necessary to prepare respective ES cell strains and stable cell strains for a clone, and the cell can be isolated by freely using a desired ES cell.

(5) Since a gene can be transferred simply into cells other than an ES cell, specificity of the prepared first recombinant DNA can be confirmed directly in other cells. In addition, construction of this gene can be simply applied to an experiment on other cells.

### Example 4

As described above, specificity of the Nkx2.5 gene promoter and the αMHC gene promoter used in Examples has been already confirmed, and it was made clear in the actual experimental results that a cardiac muscle cell can be isolated. Further, from a viewpoint of the (5), for the purpose of directly demonstrating that a DNA transferred with two adenoviruses of Example 3 can specifically visualize a target cardiac muscle cell correctly, a mouse primary cardiac muscle cultured cell was infected with these two adenoviruses.

A cardiac muscle was taken out from a neonatal mouse on day 1 after birth, and this was digested with collagenase to separate a cardiac muscle cell into a single cell, and was seeded on a culturing dish, followed by culturing.

This procedure of culturing a primary cardiac muscle cell was fundamentally performed according to the method described in Khalid M A et al. Circ. Res. 72, p725-736(1993), and Wang L et al. Circ. Res. 79, p79-85(1996). The cardiac muscle cell on day 2 after such the culturing was infected with Ad.Nkx2.5-Cre and Ad.CA-loxP-Neo-EGFP, respectively, at MOI of 5, this was observed under a fluorescent microscope 72 hours after infection, and the cardiac muscle cell with a gene transferred therein was visualized as EGFP-positive. When the cardiac muscle cell was infected with Ad.αMHC-Cre and Ad.CA-loxP-Neo-EGFP similarly according to the same protocol, the cardiac muscle cell with a gene transferred therein was visualized as EGFP-positive. Other several kinds of cultured cells (Hela human uterus cervical cancer cell, MKN28 human stomach cancer cell, LL2 mouse lung cancer cell, LM8 mouse bone sarcoma cell etc.) other than a cardiac muscle were infected with these two adenovirus vectors, and specificity thereof was tested, but such the expression of EGFP was not seen with a fluorescent microscope in all cells. It was directly demonstrated that, when the first recombinant DNA and the second recombinant DNA used in Examples 1, 2 and 3 are transferred, they clearly target only a cardiac muscle cell and visualize it with EGFP like this.

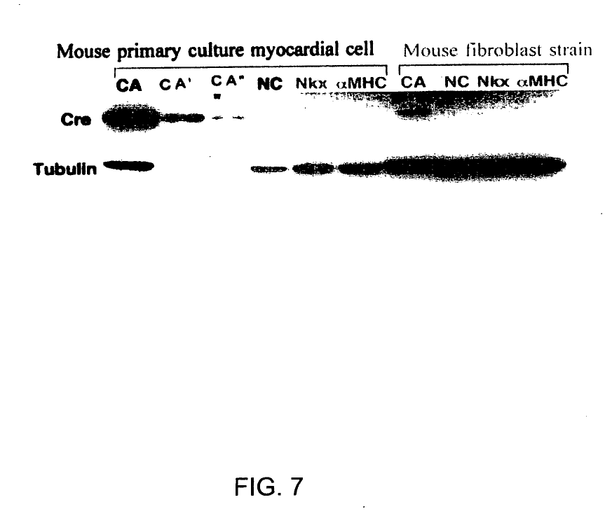

In addition, an experiment was performed in order to investigate promoter activity of a CA promoter as a constitutive strong expression promoter, and a Nkx2.5 promoter and an αMHC promoter of a tissue-specific gene. That is, Western blotting analysis (Cre antibody) was performed using a mouse primary cultured cardiac muscle cell and a NIH3T3 mouse fibroblast strain infected with the following adenovirus vectors at an infection efficacy (MOI) of 30 or 500 (see FIG. 7). CA was infected with Ad.CA-Cre, and 5 μg of a protein was electrophoresed, CA′ was infected with Ad.CA-Cre, and 0.5 μg of a protein was electrophoresed, CA″ was infected with Ad.CA-Cre, and 0.1 g of a protein was electrophoresed, NC was a negative control not infected with Ad, and 5 μg of a protein was electrophoresed, Nkx2.5 was infected with Ad.Nkx2.5-Cre, and 5 μg of a protein was electrophoresed, αMHC was infected with Ad.αMHC-Cre, and 5 μg of a protein was electrophoresed, and Tublin (protein secreted in a cardiac muscle cell or a fibroblast) was an endgeneous control, and was detected using an anti-Tublin antibody. From FIG. 7, regarding a CA promoter, recombinase Cre was detected in 10-fold diluted CA′ and 50-fold diluted CA″ in addition to the case where 5 μg of a protein was used and, regarding both of Nkx2.5 promoter and αMHC promoter, a very small amount was only detected in 5 μg of any protein. From this, it can be seen that promoter activity of a Nkx2.5 promoter and an αMHC promoter of a tissue-specific gene is very weak, and a selective marker such as EGFP is expressed only by the present invention.

### INDUSTRIAL FIELD OF APPLICABILITY

The method for selectively isolating or visualizing a target cell differentiated from an embryonic stem cell is not only widely useful in embryology, regeneration medicine and other molecular biology study by using various isolated cells and tissues, but also very useful in development of future regeneration medicine of various inveterate diseases including cardiac infarct and cerebral infarction. In addition, since a cell which has been further differentiated from a permanently and constitutively differentiated target cell can be also labeled and visualized, it becomes possible to observe and analysis a differentiated line and a tissue line on a culturing dish. Selective isolation or visualization of a target cell differentiated from an embryonic stem cell can be performed further simply by using a kit for isolation or visualization.

The present invention is not limited to the aforementioned Examples as far as included in the substantial scope of the invention.

1. A method for selectively isolating or visualizing a target cell differentiated from an embryonic stem cell, which comprises transferring a first recombinant DNA in which a first promoter, a gene having recombinase recognition sequences on both ends, and a selective marker gene of a target cell differentiated from an embryonic stem cell are arranged in this order from a 5′ side, and the first promoter makes the selective marker gene express, and a second recombinant DNA in which a second promoter specifically expressing in a target cell differentiated from an embryonic stem cell, and a recombinase-expressing gene are arranged in this order from a 5′ side, respectively, into an embryonic stem cell.

2. The method for selectively isolating or visualizing a target cell differentiated from an embryonic stem cell according to claim 1, wherein the recombinase recognition sequence is loxP.

3. The method for selectively isolating or visualizing a target cell differentiated from an embryonic stem cell according to claim 1, where the first promoter is a constitutive strong expression promoter.

4. The method for selectively isolating or visualizing a target cell differentiated from an embryonic stem cell according to claim 3, wherein the constitutive strong expression promoter is a CMV promoter or a CA promoter.

5. The method for selectively isolating or visualizing a target cell differentiated from an embryonic stem cell according to claim 1, wherein the selective marker gene is a fluorescent protein gene.

6. The method for selectively isolating or visualizing a target cell differentiated from a embryonic stem cell according to claim 1, wherein the recombinase-expressing gene is a recombinase Cre-expressing gene.

7. The method for selectively isolating or visualizing a target cell differentiated from an embryonic stem cell according to claim 1, wherin the second promoter is a Nkx2.5 gene promoter or an αMHC gene promoter.

8. The method for selectively isolating or visualizing a target cell differentiated from an embryonic stem cell according to claim 1, wherein transfer of the first recombinant DNA into an embryonic stem cell is performed using a first vector for transferring a gene.

9. The method for selectively isolating or visualizing a target cell differentiated from an embryonic stem cell according to claim 8, wherein the first vector for transferring a gene is a virus vector.

10. The method for selectively isolating or visualizing a target cell differentiated from an embryonic stem cell according to claim 9, wherein the virus vector is an adenovirus vector.

11. The method for selectively isolating or visualizing a target cell differentiated from an embryonic stem cell according to claim 1, wherein transfer of the second recombinant DNA into an embryonic stem cell is performed using a second vector for transferring a gene.

12. The method for selectively isolating or visualizing a target cell differentiated from an embryonic stem cell according to claim 11, wherein the second vector for intruding a gene is a virus vector.

13. The method for selectively isolating or visualizing a target cell differentiated from an embryonic stem cell according to claim 12, wherein the virus vector is an adenovirus vector.

14. An embryonic stem cell in which the first recombinant DNA as defined in claim 1 is transferred.

15. The embryonic stem cell in which the second recombinant DNA as defined in claim 1 is transferred.

16. The embryonic stem cell in which the first recombinant DNA and the second recombinant DNA as defined in claim 1 are transferred, respectively.

17. The embryonic stem cell according to any one of claim 14 to claim 16, wherein the embryonic stem cell is derived from a mouse.

18. A first vector for transferring a gene, which comprises the first recombinant DNA as defined in claim 8.

19. The first vector for transferring a gene according to claim 18, which is a virus vector.

20. The first vector for transferring a gene according to claim 19, wherein the virus vector is an adenovirus vector.

21. A second vector for transferring a gene, which comprises the second recombinant DNA as defined in claim 11.

22. The second vector for transferring a vector according to claim 21, which is a virus vector.

23. The second vector for transferring a gene according to claim 22, wherein the virus vector is an adenovirus vector.

24. A kit for isolation or visualization used in a method for selectively isolating or visualizing a target cell differentiated from an embryonic stem cell, which comprises the first vector for transferring a gene as defined in claim 18, and the second vector for transferring a gene as defined in claim 21.

25. The kit for isolation or visualization used in a method for selectively isolating or visualizing a target cell differentiated from an embryonic stem cell according to claim 24, wherein the first vector for transferring a gene and the second vector for transferring a gene are a virus vector.

26. The kit for isolation or visualization used in a method for selectively isolating or visualizing a target cell differentiated from an embryonic stem cell according to claim 25, wherein the virus vector is an adenovirus vector.

27. The kit for isolation of visualization used in a method for selectively isolating or visualizing a target cell differentiated from an embryonic stem cell, which comprises the embryonic stem cell as defined in claim 14, and the second vector for transferring a gene as defined in claim 21.

28. The kit for isolation or visualization used in a method for selectively isolating of visualizing a target cell differentiated from an embryonic stem cell according to claim 27, wherein the second vector for transferring a gene is a virus vector.

29. The kit for isolation or visualization used in a method for selectively isolating or visualizing a target cell differentiated from an embryonic stem cell according to claim 28, wherein the virus vector is an adenovirus vector.

30. The kit for isolation or visualization used in a method for selectively isolating or visualizing a target cell differentiated from an embryonic stem cell, which comprises the first vector for transferring a gene as defined in claim 18, and the embryonic stem cell as defined in claim 15.

31. The kit for isolation or visualization used in a method for selectively isolating or visualizing a target cell differentiated from an embryonic stem cell according to claim 30, wherein the first vector for transferring a gene is a virus vector.

32. The kid for isolation or visualization used in a method for selectively isolating or visualizing a target cell differentiated from an embryonic stem cell according to claim 31, wherein the virus vector is an adenovirus vector.

33. A cell obtained by the method for selectively isolating or visualizing a target cell differentiated from an embryonic stem cell as defined in claim 1.

34. The cell according to claim 33, wherein the cell is a cell obtained by using a Nkx2.5 gene promoter as the second promoter.

35. The cell according to claim 33, wherein the cell is a cardiac muscle cell obtained by using an αMHC gene promoter as the second promoter.

36. A tissue, which comprises the cell as defined in claim 33.

37. A method for treating a disease, which comprises using the cell as defined in claim 33 and/or the tissue as defined in claim 36.

38. The method for treating a disease according to claim 37, wherein the disease is a cardiac disease.
